(19) United States
(12) Patent Application Publication
(10) Pub. No.: US 2005/0014845 A1
(43) Pub. Date: Jan. 20, 2005

(54) Ambroxol for the treatment of epilepsy

(75) Inventors: Wolfram Gaida, Ingelheim (DE); Klaus Klinder, Oggelshausen (DE); Thomas Weiser, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/888,364

(22) Filed: Jul. 9, 2004

(30) Foreign Application Priority Data
Jul. 16, 2003 (DE) 103 32 473.9

(51) Int. Cl. A61K 31/498; A61K 31/137

(52) U.S. Cl. 514/650; 514/355; 514/248; 514/249

(57) ABSTRACT

A method of treating epilepsy, with the exception of epileptic states, in a patient in need thereof, the method comprising administering to the patient ambroxol or a pharmacologically acceptable salt thereof.

### RELATED APPLICATIONS

This application claims priority to German patent application No. DE 103 32 473.9, filed Jul. 16, 2003, which is hereby incorporated by reference in its entirety.

### FIELD OF THE INVENTION

The invention relates to the use of ambroxol and the pharmacologically acceptable salts thereof for preparing a pharmaceutical composition for the treatment of epilepsy.

### BACKGROUND OF THE INVENTION

The active substance ambroxol (trans-4-(2-amino-3,5-dibromobenzylamino)cyclohexanol) is a known local anesthetic, antitussive and expectorant. In addition, ambroxol's effect as a sodium channel blocker is described in the literature (Society for Neuroscience Abstracts, 2000, Vol. 26, No. 1-2). The potential activity of sodium channel blockers as agents for treating epilepsy is also known from the prior art (H. Choi and M. J. Morrell, 2003, Expert Opin Pharmacother 4(2), 243-51).

Known sodium channel blockers which are used to treat epilepsy are, however, limited in their efficacy. In addition, they frequently have undesirable side effects (Perucca et al. 2000, Epilepsy Res 41, 107-139).

It is also known from the prior art that calcium channel blockers, such as, for example, gabapentin, which inhibits the calcium channels directly or indirectly by modulating the expression of the alpha2delta subunit (see K. J. Alden and J. Garcia, 2001, J Pharmacol Exp Ther 297(2), 727-35; A. Vega-Hernandez and R. Felix, 2002, Cell Mol Neurobiol 22(2), 185-90), may be suitable for treating epilepsy.

AMPA (α-amino-3-hydroxy-5-methyl-4-isoxazolepropionate) receptor antagonism has also been proposed as a possible therapeutic approach to the treatment of epilepsy (e.g., Meldrum 1994, Neurology 44(11 Suppl. 8), p.14-23).

The aim of the present invention is to provide an active substance for the treatment of idiopathic and symptomatic epilepsy which has no or only negligible central nervous and cardiovascular side effects. In addition, the active substance thus prepared should be suitable for oral administration and thus have good bioavailability.

### DESCRIPTION OF THE INVENTION

Surprisingly, ambroxol exhibits very good activity in the treatment of idiopathic and symptomatic epilepsy, for example, in the treatment of focal or generalized epileptic attacks. At a pharmacologically effective dose there are no central nervous and cardiovascular side-effects. Surprisingly, in addition to blocking the sodium channels, ambroxol also exhibits very good effects as a calcium channel blocker and as an AMPA receptor antagonist, which as additional mechanisms provide an enhanced antiepileptic activity.

The invention therefore relates to the use of ambroxol or one of the pharmacologically acceptable salts thereof for preparing a pharmaceutical composition for the treatment of epilepsy, with the exception of the epileptic state.

Preferably ambroxol or one of the pharmacologically acceptable salts thereof is used to prepare a pharmaceutical composition for the treatment of forms of epilepsy with focally starting attacks, most preferably forms of epilepsy with focally starting idiopathic or symptomatic attacks.

It is particularly preferred to use ambroxol or one of the pharmacologically acceptable salts thereof for preparing a pharmaceutical composition for the treatment of forms of epilepsy with generalized attacks, for example, idiopathic, cryptogenic, or symptomatic, or symptomatic generalized forms of epilepsy, most preferably idiopathic generalized attacks.

The present invention further relates to the use of an orally administered pharmaceutical composition containing ambroxol or one of the pharmacologically acceptable salts thereof, preferably in the form of a tablet.

The use of ambroxol is particularly preferred, the ambroxol being used in a daily dose of 30 mg to 4000 mg, preferably 150 mg to 3000 mg, more preferably 350 mg to 2500 mg, and most preferably 500 mg to 2000 mg.

The present invention also relates to a pharmaceutical composition containing ambroxol and one or more active substances selected from the group consisting of sodium channel blockers, calcium channel antagonists, preferably N subtype blockers, P/Q subtype blockers, gallopamil, verapamil, diltiazem, nifedipine, and amlodipine, glutamate receptor antagonists, preferably ketamine and memantine, 2,3-benzodiazepines, preferably Gyki compounds, quinoxaline-diones, preferably NBQX, anticonvulsants, preferably gabapentin, pregabalin, carbamazepine, lamotrigin, topiramate, phenyloin, and levitiracetam, and potassium channel openers, preferably retigabin.

Preferably, ambroxol or one of the pharmacologically acceptable salts thereof is used in combination with one or more other active substances, selected from the group consisting of sodium channel blockers, calcium channel antagonists, preferably N subtype blockers, P/Q subtype blockers, gallopamil, verapamil, diltiazem, nifedipine, and amlodipine, glutamate receptor antagonists, preferably ketamine and memantine, 2,3-benzodiazepines, preferably Gyki compounds, quinoxaline-diones, preferably NBQX, anticonvulsants, preferably gabapentin, pregabalin, carbamazepine, lamotrigin, topiramate, phenyloin, and levitiracetam, and potassium channel openers, preferably retigabin.

The name ambroxol within the scope of the present invention denotes both the base ambroxol, and also the solvates or hydrates thereof, preferably the base ambroxol.

Acids suitable for forming salts of ambroxol are, for example, hydrochloric acid, hydrobromic acid, sulfuric acid, phosphoric acid, nitric acid, oxalic acid, malonic acid, fumaric acid, maleic acid, tartaric acid, citric acid, ascorbic acid, and methanesulfonic acid, preferably hydrochloric acid.

The effects of ambroxol according to the invention will be illustrated by the Examples that follow. These serve merely to illustrate the invention and are not to be regarded as limiting it.

Ambroxol inhibits various neuronal sodium channel subtypes, the half-maximal inhibition of centrally expressed channels being at 111 μM (Weiser and Wilson 2002, Mol Pharmacol 62, 433-438).

Voltage-dependent calcium channels play an important role in neurotransmission. The blocking of these channels has been described as being helpful for the treatment of epilepsy (K. J. Alden and J. Garcia, 2001, J Pharmacol Exp Ther 297(2), 727-35; A. Vega-Hernandez and R. Felix, 2002, Cell Mol Neurobiol 22(2), 185-90).

It has been found, surprisingly, that ambroxol also blocks voltage-dependent calcium channels in neuron cultures from rats in concentrations of 10 μM to 1000 μM. Neurons were dissected from posterior root ganglia of adult rats and placed in short-term culture. The cells were investigated electrophysiologically by the Patch-Clamp method (voltage terminal), and the flow of current through voltage-dependent calcium channels was measured after electrical stimulation (voltage jumps from −80 mV to 0 mV holding potential for 50 ms) in the presence and absence of ambroxol.

Ionotropic glutamate receptors of the AMPA subtype are also essential for the excitatory neurotransmission. The blocking of AMPA receptors has been described as being helpful for the treatment of epilepsy (e.g., Meldrum 1994, Neurology 44 (11 Suppl. 8), p.14-23).

In HEK 293 cells which express heterologously human GluR1/2 receptors, ambroxol surprisingly inhibits glutamate-induced membrane currents in concentrations ranging from 30-1000 μM. HEK 293 cells which expressed functionally recombinant human GluR1/2 receptors were electrophysiologically investigated by the Patch-Clamp method (voltage terminal). The administration of 1 mM glutamate (for 1 s at a holding potential of −80 mV) induced membrane currents which were inhibited by the joint administration of ambroxol.

Ambroxol may be used on its own or in combination with other pharmacologically active substances. Suitable preparations include, for example, tablets, capsules, suppositories, solutions, elixirs, emulsions, or dispersible powders. Suitable tablets may be obtained, for example, by mixing the active substance(s) with known excipients, for example, inert diluents such as calcium carbonate, calcium phosphate, or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example, collidone or shellac, gum arabic, talc, titanium dioxide, or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol, or sugar and a flavor enhancer, e.g., a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g., with the addition of preservatives such as p-hydroxybenzoates, or stabilizers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

A therapeutically effective daily dose is in the range from 30 mg to 4000 mg, preferably 150 mg to 3000 mg, more preferably 350 mg to 2500 mg, and most preferably 500 mg to 2000 mg, in adults.

The Examples which follow illustrate the present invention without restricting its scope:

Examples of Pharmaceutical Formulations

| | | |
|---|---|---|
| A) | Tablets | per tablet (mg) |

| | |
|---|---|
| ambroxol | 800 |
| lactose | 140 |
| maize starch | 240 |
| polyvinylpyrrolidone | 20 |
| magnesium stearate | 10 |

Ambroxol, lactose and some of the maize starch are mixed together. The mixture is screened, then moistened with a solution of polyvinylpyrrolidone in water, kneaded, wet-granulated, and dried. The granules, the remaining maize starch, and the magnesium stearate are screened and mixed together. The mixture is compressed to produce tablets of suitable shape and size.

| | | |
|---|---|---|
| B) | Tablets | per tablet (mg) |

| | |
|---|---|
| ambroxol | 800 |
| maize starch | 190 |
| lactose | 55 |
| microcrystalline cellulose | 35 |
| polyvinylpyrrolidone | 20 |
| sodium-carboxymethyl starch | 30 |
| magnesium stearate | 10 |

Ambroxol, some of the corn starch, lactose, microcrystalline cellulose, and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| | | |
|---|---|---|
| C) | Coated tablets | per coated tablet (mg) |

| | |
|---|---|
| ambroxol | 500 |
| maize starch | 45 |
| lactose | 30 |
| polyvinylpyrrolidone | 5 |
| magnesium stearate | 5 |

Ambroxol, maize starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 11 mm are compressed in a tablet-making machine. The tablet cores thus produced are coated in known manner with a covering consisting essentially of sugar and talc. The finished coated tablets are polished with wax.

| | | |
|---|---|---|
| D) | Capsules | per capsule (mg) |

| | |
|---|---|
| Ambroxol | 250 |
| maize starch | 268.5 |
| magnesium stearate | 1.5 |

Ambroxol and maize starch are mixed and moistened with water. The moist mass is screened and dried. The dry granules are screened and mixed with magnesium stearate. The finished mixture is packed into size 1 hard gelatine capsules.

| | |
|---|---|
| E) | Parenteral solution |

| | |
|---|---|
| Ambroxol | 500 mg |
| Citric acid monohydrate | 100 mg |
| sodium hydroxide | 35 mg |
| mannitol | 1500 mg |
| water for inj. | 50 mL |

The ambroxol is dissolved in water at its own pH or optionally at pH 5.5 to 6.5 and mannitol is added to make it isotonic. The solution obtained is filtered free from pyrogens and the filtrate is transferred under aseptic conditions into injection vials which are then sealed with rubber stoppers and autoclaved.

| | |
|---|---|
| F) | Suppositories |

| | |
|---|---|
| Ambroxol | 450 mg |
| solid fat | 1650 mg |

The hard fat is melted. At 40° C. ambroxol is homogeneously dispersed therein. The mixture is cooled to 38° C. and poured into slightly chilled suppository moulds.

| | |
|---|---|
| G) | Oral solution |

| | | |
|---|---|---|
| ambroxol | 150 | mg |
| hydroxyethylcellulose | 50 | mg |
| sorbic acid | 5 | mg |
| sorbitol (70%) | 600 | mg |
| glycerol | 200 | mg |
| flavoring | 15 | mg |
| water | to 10 | mL |

Distilled water is heated to 70° C. Hydroxyethylcellulose is dissolved therein with stirring. After the addition of sorbitol solution and glycerol, the mixture is cooled to ambient temperature. At ambient temperature sorbic acid, flavoring, and ambroxol are added. To eliminate air from the suspension, it is evacuated with stirring.

1. A method of treating epilepsy, with the exception of epileptic states, in a patient in need thereof, the method comprising administering to the patient ambroxol or a pharmacologically acceptable salt thereof.

2. The method according to claim 1, wherein the epilepsy is idiopathic epilepsy.

3. The method according to claim 1, wherein the epilepsy is symptomatic epilepsy.

4. The method according to claim 1, wherein the ambroxol or a pharmacologically acceptable salt thereof is administered orally to the patient.

5. The method according to one of claims 1 to 4, wherein the ambroxol or a pharmacologically acceptable salt thereof is administered to the patient in a daily dose of 30 mg to 4000 mg.

6. The method according to one of claims 1 to 4, wherein one or more additional active substances selected from sodium channel blockers, calcium channel antagonists, glutamate receptor antagonists, quinoxaline-diones, anticonvulsants, and potassium channel openers are administered to the patient before, after, or simultaneously with the ambroxol or a pharmacologically acceptable salt thereof.

7. A pharmaceutical composition comprising:
(a) ambroxol or a pharmacologically acceptable salt thereof; and
(b) one or more additional active substances selected from sodium channel blockers, calcium channel antagonists, glutamate receptor antagonists, quinoxaline-diones, anticonvulsants, and potassium channel openers.
